(19) United States
(12) Patent Application Publication
(10) Pub. No.: US 2016/0050866 A1
(43) Pub. Date: Feb. 25, 2016

(54) MICROPROPAGATION AND PLANT REGENERATION SYSTEMS FOR ARUNDO DONAX AND OTHER MONOCOTS

(71) Applicant: The Institute for Advanced Learning and Research, Danville, VA (US)

(72) Inventors: Yinghui Dan, Raleigh, NC (US); Anni Kekkonen, Helsinki (FI); N. Faith Campbell, Ridgeway, VA (US)

(21) Appl. No.: 14/463,925

(22) Filed: Aug. 20, 2014

(51) Int. Cl. A01H 4/00; A01G 1/00; A01H 5/10

(52) U.S. Cl. CPC A01H 4/005; A01H 5/10; A01G 1/001

(57) ABSTRACT

Systems, methods and media formulations for high-quality and large-scale micropropagation without callus phase and plant regeneration from callus of graminaceous monocot plants such as Arundo, corn and wheat involving composite meristem explants, leaf explants and other explants have been developed. Graminaceous plants and plantlets propagated and regenerated by these systems, methods, and medium formulations are also described herein.

### FIELD OF THE INVENTION

The invention relates generally to media, systems, and methods for large-scale micropropagation and plant regeneration systems for commercial monocot crop plants such as Arunndo donax, corn and wheat.

### BACKGROUND

Giant reed (Arundo donax L.) is one of the most promising species for energy, cellulose paste, and second-generation biofuel production because of its perennial nature, ease of adaptation to different environmental conditions, high levels of biomass production, and low annual input requirements after establishment. It is also useful in the control of soil erosion and an effective candidate for phytoremediation of nitrate- or heavy metal contaminated water and soils, as it also has a robust root system, provides ground cover, and retains living stems during winter. It is exclusively and vegetatively-propagated from fragments of stems and rhizomes, due to its reproductive sterility, which is time-consuming and cost expensive, limiting large-scale cultivation. To alleviate these negative aspects, an efficient in vitro propagation system is required to speed up Arundo breeding and biofuel processing.

### SUMMARY

Described herein are systems, methods, and media formulations for large-scale production of a graminaceous plant and methods for regenerating a graminaceous plant from a callus. To date there is no efficient in vitro micropropagation method using in vitro composite meristem containing root, apical and intercalary meristems as explants. Furthermore, there is no efficient plant regeneration method via callus phase using composite meristem, leaf and root as explants. This lack of an efficient propagation system restricts Arundo breeding potential. In addition, genetic manipulation methods, including mutagnesis, for many plant species usually require a plant regeneration system based on totipotent calli. Therefore, both a large-scale, high throughput production (micropropagation) system and a plant regeneration system via a callus phase, for the breeding and genetic manipulation of Arundo and other monocots, including corn and wheat (e.g., cereal crops), have been developed and are described herein. Methods for large-scale production of a graminaceous plant involve direct regeneration using a composite meristem explant (i.e., regeneration without a callus phase). Methods for regenerating a graminaceous plant from a callus involve indirect regeneration via a callus phase using one of the following explants: composite meristem explant, leaf explant and root explant. The experimental results described herein in the Examples section include data pertaining to corn, wheat and Arundo. In one example of the Arundo data presented herein, a high throughput system was developed for multiple shoot induction and rooting for Arundo genotypes, including genotype CMT1 and two commercial cultivars of Arundo donax (cvs. Peppermint Stick and Variegata) using composite meristem explants explants. Using this system, 10- to 36-fold shoot production rates were obtained within 3 to 4 weeks for the three genotypes tested. One hundred percent of shoots produced roots within 2 to 3 weeks (Table 1, Table 2 and FIG. 2). Using this method, massive shoot production (up to more than 60 shoots/buds) was obtained from a single shoot for the three genotypes (FIG. 3). Regarding the corn data, methods for regenerating a corn plant or plantlet from a callus are described, specifically indirect regeneration methods via callus phase using composite meristem explants (Table 13 and Table 14) as well as indirect regeneration methods via callus phase using leaf explant (Table 15). Regarding the wheat data, methods for large-scale production of a wheat plant or plantlet using a composite meristem explant (i.e., regeneration without a callus phase) are described (Table 16). Also, methods for regenerating a wheat plant or plantlet from a callus are described, specifically indirect regeneration methods via a callus phase using leaf explant (Table 17 and Table 18).

Accordingly, described herein is a method for large-scale production of a graminaceous plant (e.g., Arundo, corn and wheat) that includes the steps of: isolating a plurality of composite meristem explants from at least one of in vitro graminaceous plant culture and seedlings under sterile conditions; culturing the plurality of composite meristem explants in multiple shoot induction medium containing at least one plant growth hormone under sterile conditions such that at least one of multiple shoots and multiple buds grow from each composite meristem explant; culturing the at least one of multiple shoots and multiple buds in basal medium lacking plant growth hormones under sterile conditions such that roots grow from the shoots resulting in a plurality of plantlets; and transferring the plurality of plantlets to a substrate comprising a carbohydrate-free medium that enables growth of the plantlets under non-sterile conditions, and propagating the plantlets. In the method, the at least one of multiple shoots and multiple buds can include up to approximately 60 shoots per explant, up to approximately 60 buds per explant, or up to approximately 60 shoots and buds per explant. The substrate can be any suitable substrate, e.g., at least one Peat plug. A vacuum system can be used for culturing in one or both of the steps of: culturing the plurality of composite meristem explants in multiple shoot induction medium and culturing the at least one of multiple shoots and multiple buds in basal medium. The method can further include the step of transplanting the plurality of plantlets into soil. In a typical embodiment, the method provides a rooting efficiency in the range of about 90% to about 100%.

Also described herein is a plurality of graminaceous plantlets produced according to this method.

Further described herein is a method for large-scale production of wheat plantlets that includes the steps of: isolating a plurality of composite meristem explants from at least one of in vitro plantlet culture and germinated seedlings under sterile conditions; culturing the plurality of composite meristem explants in multiple shoot induction medium containing at least one plant growth hormone under sterile conditions such that at least one of multiple shoots and multiple buds grow from each composite meristem explant; and culturing the at least one of multiple shoots and multiple buds in basal medium comprising sucrose, vitamins and a cytokinin under sterile conditions such that roots grow from the shoots resulting in a plurality of wheat plantlets. The method can further include sterilizing and germinating a plurality of wheat seeds resulting in germinated seedlings.

Additionally described herein is a plurality of wheat plantlets produced according to this method.

Yet further described herein is A method for regenerating a graminaceous plantlet (e.g., Arundo) from a callus that includes the steps of: isolating an explant from at least one of in vitro graminaceous plant culture and seedlings under sterile conditions; culturing the explant in embryonic callus induction medium under sterile conditions such that a callus is produced and buds grow from the callus; culturing the callus in plant regeneration medium for callus under sterile conditions such that multiple shoots grow from the callus; and culturing the shoots and buds in basal medium under sterile conditions such that roots grow from the shoots resulting in production of a plantlet. The explant can be, for example, a composite meristem explant, a root explant, and/or a leaf explant. The method can further include subjecting the callus and the plantlet to chemical mutagenesis and genetic modification via Agrobacterium-mediated and biolistic transformation.

Also described herein is a graminaceous plantlet regenerated by this method.

Still further described herein is a method for regenerating wheat plantlets from calli that includes the steps of: sterilizing and germinating a plurality of wheat seeds resulting in a plurality of germinated seedlings; isolating leaf explants from the germinated seedlings under sterile conditions; culturing the leaf explants in embryonic callus induction medium under sterile conditions such that calli are produced and buds grow from the calli; culturing the calli in plant regeneration medium for callus under sterile conditions such that shoots grow from the calli; and culturing the shoots and buds under conditions such that roots grow from the shoots resulting in production of wheat plantlets. The method can further include subjecting the calli and the wheat plantlets to chemical mutagenesis and genetic modification via Agrobacterium-mediated and biolistic transformation.

Also described herein is a wheat plantlet regenerated by this method.

Additionally described herein is a method for regenerating corn plantlets from calli that includes the steps of: isolating explants under sterile conditions, wherein the explants are at least one of composite meristem explants and leaf explants from at least one of: in vitro corn plant culture and seedlings; culturing the explants in embryonic callus induction medium for callus under sterile conditions such that calli are produced and buds grow from the calli; culturing the calli in plant regeneration medium for callus under conditions such that shoots grow from the calli; and culturing the shoots and buds under conditions such that roots grow from the shoots resulting in production of corn plantlets. The method can further include subjecting the corn plantlets to chemical mutagenesis and genetic modification via Agrobacterium-mediated and biolistic transformation.

Still further described herein is a corn plantlet regenerated by this method.

Systems that include one or more of the explants, devices, culture reagents, etc., described herein encompassed by the invention. Such a system can include, for example, one or more explants and tissue culture reagents, including plastic trays or dishes and one or more culture medium formulations.

Unless otherwise defined, all technical terms used herein have the same meaning as commonly understood by one of ordinary skill in the art to which this invention belongs.

As used herein, the term “graminaceous plant” means a grass-like plant, with hollow jointed stems and long narrow leaves, having the ability to produce tillers from the bottom of an axillary meristem. Arundo, corn and wheat species are examples of graminaceous plants.

By the term “shoot” is meant the aerial portion of a plant, including stem, branches, and leaves.

As used herein, the term “bud” means a small swelling or protuberance on a stem or branch of a plant, containing the undeveloped shoot and leaf.

As used herein the term “plantlet” refers to a young or small plant with roots.

By the phrase “composite meristem explant” is meant a plant segment containing at least a portion of apical meristem, at least a portion of intercalary meristem and at least a portion of root meristem.

By the term “micropropagation” is the art and science of plant multiplication under aseptic conditions. The process usually includes explant sterilization, callus induction and propagation, plantlet regeneration, shoot multiplication, rooting, and acclimation.

As used herein, the term “large-scale” generally means involving great numbers or quantities, and/or large in comparison with others of the same general class. In a typical embodiment, a “large-scale” regeneration system is one with an efficiency of at least more than 5-fold (e.g., 10-fold to 100-fold) shoots and/or bud production from a single explant.

The term “high-throughput” means the capability to produce plantlets and/or plants on a large scale in a relatively short time.

By the term “chemical mutagenesis” is meant any technique or method used to induce or introduce a mutation into a nucleic acid (e.g., a plant gene).

The term “about” as used herein when referring to a measurable value such as concentration, time, temperature, etc. is meant to encompass variations of +/−5% of the specified amount.

Although media, systems and methods similar or equivalent to those described herein can be used in the practice or testing of the present invention, suitable media, systems and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a photograph of a plant segment containing three types of meristems, including 1) apical, 2) intercalary and 3) root meristems. These three segments combined constitute a composite meristem explant.

FIG. 2 is a series of photographs showing massive production of Arundo plants within 1 to 2 wks after culture for genotype CMT1 through composite meristem explant.

FIG. 3 is a photograph showing regeneration of massive shoots and buds (approximately more than 60) from a single composite meristem explant of Arundo donax.

FIG. 4 is a series of photographs of an Arundo production system using a high throughput micropropagation system as described herein. FIG. 5 is a photograph of a device for culture medium transfer.

FIG. 6 is a photograph of organogenic calli derived from composite meristem explants for genotype CMT1.

FIG. 7 is a photograph of approximately 164 shoots/buds obtained from a single callus unit and plant regeneration for genotype CMT1.

FIG. 8 is a photograph of embryo-like structure in medium EC-11 from calli derived from composite meristem explants for genotype CMT1.

FIG. 9 is a photograph of embryogenic-like callus induction, derived from root explants for genotype CMT1.

FIG. 10 is a photograph of bud primordium formation from calli derived from root explants for genotype CMT1.

FIG. 11 is a photograph of bud primordium formation from calli derived from leaf explants for genotype CMT1.

FIG. 12 is a photograph of embryogenic-like callus induction derived from leaf explants for genotype CMT1.

FIG. 13 is a photograph of plant and shoot regeneration via callus phase from composite meristem explants in corn, genotype H99.

FIG. 14 is a photograph of plant and shoot regeneration from leaf explants in corn, genotype H99.

FIG. 15 is a photograph of shoot regeneration from composite meristem explants in wheat, genotype Bobwhite.

FIG. 16 is a photograph of callus and bud primordium formation from leaf explants in wheat, genotype Bobwhite.

FIG. 17 is a photograph of massive shoot and bud regeneration (approximately 94 buds and shoots) from a single leaf explant of Arundo, Genotype CMT1.

FIG. 18 is a photograph of massive plant regeneration (total more than 120 plants, shoot and buds) from single leaf explant of Arundo, genotype CMT 1.

### DETAILED DESCRIPTION

Described herein are novel media formulations, methods and systems for high-quality and large-scale micropropagation and plant regeneration from callus of graminaceous monocot plants such as Arundo, corn and wheat involving composite meristem explants and other explants. Graminaceous plants and plantlets propagated and regenerated by these systems, methods and media formulations are also described herein.

### Plant Culture Methods

Methods involving conventional plant and plant cell culture techniques are described herein. Such techniques are generally known in the art and are described in detail in methodology treatises such as Plant Tissue Culture, by Margit Laimer and Waltraud Rucker, 2003, 1^{st} ed., Springer, New York, N.Y.; and Plant Cell Culture: Essential Methods, by Michael R. Davey and Paul Anthony, 2010, Wiley, Hoboken, N.J. Also see, for example, US patent application publication no. 2008-0282424 A1 (U.S. patent application Ser. No. 11/800,719) and PCT application no. WO2010/011717, each incorporated herein by reference.

### Explants

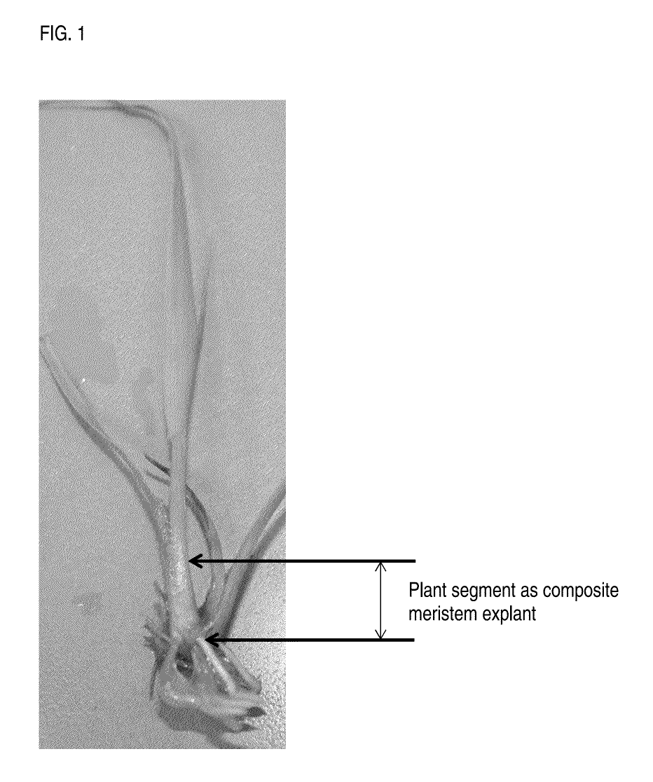

Referring to FIG. 1, this plant segment, also referred to herein as a “composite meristem explant,” contains three types of meristems, including 1) apical, 2) intercalary and 3) root meristems as meristem explant. Typical explants known in the art are either a shoot tip that only contains apical meristem or axillary buds that only contain intercalary meristem (i.e., a single meristem explant). In contrast, a composite meristem explant as described herein contains at least a portion of (e.g., a piece or fragment of, or a full length apical, intercalary or root meristem) each of: apical meristem, intercalary meristem and root meristem. Use of a composite meristem explant as described herein promotes plant regeneration due to the developmental interaction of the three meristems, compared to a single meristem explant (such as shoot tip or axillary bud explants). The results described in the Examples section demonstrate a significantly 3.6 to 14.4-fold higher propagation efficiencies for our micro propagation system and 10 to more than 100-fold higher regeneration efficiencies for our regeneration systems via callus phase when using a composite meristem explant compared with previously published Arundo micro propagation and regeneration methods (Cavallaro et al., 2008) Marton and Czako, 2004, 2007, 2011; Takahashi et al., 2010).

In some methods of regenerating a graminaceous plant from a callus (indirect regeneration via a callus phase), leaf explants and root explants are used in addition to composite meristem explants. The term “leaf explant” means a leaf from in vitro plants or seedlings not including meristem. Typically, a leaf explant is a segment approximately 0.5 cm (e.g., in the range of about 0.2 to about 1.5 cm) in length from the base of the leaf. The term “root explant” means a main root from in vitro plants or seedlings including a single root meristem. Typically, a root explant is a segment approximately 0.5 cm (e.g., in the range of about 0.2 to about 1.5 cm).

The explants as described herein can be used for the large-scale production and regeneration of any monocot. Monocotyledons, also known as monocots, are one of two major groups of flowering plants that are traditionally recognized, the other being dicotyledons, or dicots. Monocot seedlings typically have one cotyledon (seed-leaf). The true grasses, family Poaceae (Gramineae), are an economically important family. These include all the true grains including, e.g., rice, wheat and maize (the cereal crops), the pasture grasses, sugar cane, the bamboos, Arundo and other monocots.

### Methods of Large-scale Production (Propagation) of Plantlets and Plants

Described herein are highly efficient methods for large-scale and high-quality propagation of graminaceous plants such as Arundo, wheat and corn. A plant (e.g., a young plant, plantlet, full-grown plant) produced by this method, as well as one or more progeny thereof, are also described herein. In a typical method for large-scale production of a graminaceous plant, the following steps are followed: isolating a plurality of composite meristem explants from in vitro graminaceous plant culture and/or seedlings under sterile conditions; culturing the plurality of composite meristem explants in multiple shoot induction medium containing at least one plant growth hormone under sterile conditions such that multiple shoots and/or buds grow from each composite meristem explant; culturing the shoots and/or buds in basal medium lacking plant growth hormones under sterile conditions such that roots grow from the shoots resulting in a plurality of plantlets; and transferring the plurality of plantlets to a substrate (e.g., at least one Peat plug) including a carbohydrate-free medium that enables growth of the plantlets under non-sterile conditions, and propagating the plantlets. In the method, multiple shoots and/or buds typically includes approximately up to 60 shoots (e.g., 30, 40, 45, 50, 55, 60) and/or buds per explant. The graminaceous plant can be, for example, Arundo, corn or wheat. In the method, a vacuum system (e.g., a vacuum system including a pipette) can be used for efficiently handling liquid medium transfer while culturing one or more of the composite meristem explants, and the shoots and/or buds. The method can further include the step of transplanting the plurality of plantlets into soil (e.g., soil in a field, soil in a greenhouse, etc.). Typically, the method provides a rooting efficiency of about 100% (e.g, in the range of about 90% to about 100%, in the range of about 95% to about 100%, etc.).

In one example of such a method for producing Arundo plants, for example, the following steps are followed. First, isolate composite meristem explants from an in vitro plant culture. Culture 2 to 3 composite meristem explants in baby jars containing approximately 30 ml liquid of a suitable multiple shoot induction medium (e.g., MI-2, MI-3, MI-4 and MI-5) at 117.7 μmol m⁻² s⁻¹ and 24° C. under a 16 h photoperiod for approximately 2-4 weeks to produce multiple shoots and/or buds. Next, culture the multiple shoots/buds in MS basal medium without plant growth hormone at 117.7 μmol m⁻² s⁻¹ and 24° C. under a 16 h photoperiod for approximately 1-2 weeks to generate roots. Following root production, the plantlets are transferred into a suitable substrate, for example, Peat Plugs containing 50 ml of liquid root induction medium (e.g., MGM_{—}1), under non-sterile conditions at 110.2 μmol m⁻² s⁻¹ and 22° C. (range from 20° C. to 27° C.) under a 16 h photoperiod for approximately 2 weeks. By the term “photoperiod” is meant the interval in a 24-hour period during which plant culture is exposed to light.

In an embodiment in which wheat plants are being propagated on a large scale, the method can generally include the following steps. First, a plurality of composite meristem explants are isolated from in vitro plantlet culture and/or germinated seedlings under sterile conditions. Next, the plurality of composite meristem explants are cultured in multiple shoot induction medium containing at least one plant growth hormone under sterile conditions such that multiple shoots and/or buds grow from each composite meristem explant. Then, the shoots and/or buds are cultured in basal medium comprising sucrose, vitamins and a cytokinin under sterile conditions such that roots grow from the shoots resulting in a plurality of wheat plantlets. This method can further include sterilizing and germinating a plurality of wheat seeds resulting in germinated seedlings for generating the composite meristem explant.

In one example of such a method for producing wheat plants, the following steps are followed. First, sterilize and germinate seeds using the protocol set forth in Example 9. Second, isolate composite meristem explants from germinated seedlings. Next, culture the composite meristem explants in an appropriate embryonic callus induction medium (e.g., A2) or in an appropriate multiple shoot induction medium (e.g., MI-2 medium) in the dark at 26° C. for approximately 4 weeks to induce multiple buds and shoots. Then culture the multiple shoots/buds in an appropriate plant regeneration medium (e.g., ECR-3) at 117.7 μmol m−2 s−1 and 24° C. under a 16 h photoperiod for approximately 3 to 4 weeks for root growth and plant regeneration.

In a typical embodiment of the methods, composite meristem explants are isolated from an in vitro graminaceous plant culture or seedlings. An in vitro graminaceous plant culture can be any culture that a plant culture generated from in vitro tissue culture condition. Also in a typical embodiment, the pH for the liquid media formulations described herein is 5.8, but can be in the range of about 5 to about 6.5, and the culture temperature is typically around 24-26° C., but can be in the range of about 20 to 28° C. However, the media formulations described herein may have any suitable pH and temperature for the particular culturing step being performed.

An Arundo plant propagated by the methods described herein may be any variety, species and/or clone of Arundo including, but not limited to, Arundo donax. Similarly, a corn or wheat plant propagated by the methods described herein may be any variety, species and/or clone of corn or wheat, respectively. In some embodiments, a plant propagated by the methods described herein can be a hybrid of different species, varieties of a specific species, or clones of a variety.

As mentioned above, the methods described herein provide a number of advantages. For example, using the methods as described herein, the time-consuming and costs disadvantages associated with large-scale cultivation of Arundo are alleviated. The methods described herein involve a high-throughput micropropagation system with up to a 30-fold shoot multiplication rate and 100% rooting efficiency using in vitro established composite meristem explant and/or, shoot and bud cultures as explants; and 2) an efficient plant regeneration system via a callus phase from composite meristem, leaf and root explants, with up to 164 shoots generated from a single callus unit (approximately 0.5 cm in diameter), with a 100% rooting efficiency.

### Methods for Regenerating a Graminaceous Plantlet or Plant From a Callus

The regeneration methods described herein can be used to regenerate any graminaceous plantlet or plant from a callus. A typical method for regenerating a graminaceous plantlet from a callus includes: isolating an explant from an in vitro graminaceous plant culture and/or seedlings; culturing the explant in callus induction medium under sterile conditions such that a callus is produced and buds grow from the callus; culturing the callus in plant regeneration medium under sterile conditions such that multiple shoots grow from the callus; and culturing the shoots and/or buds in basal medium with or without plant growth hormones under sterile conditions such that roots grow from the shoots resulting in production of a plantlet. In this method, the graminaceous plant can be Arundo, for example. The explant can be one of: a composite meristem explant, a root explant, and a leaf explant. The method can further include subjecting the plantlet to chemical mutagenesis.

The method described above is particularly useful for regenerating Arundo. In one particular embodiment of this method for regenerating Arundo, the following steps are followed. First, isolate one or more of the following explants from an in vitro plant culture: composite meristem explants, leaf explant (approximately 0.5 cm in length from the base of leaf) and root explant (approximately 0.5 cm in length from the tip of roots). Culture each explant in suitable embryonic callus induction medium (e.g., A4, EC-1, EC-6, EC-7, EC-8, EC-9, EC-10 and EC-11) in the dark at 26° C. for approximately 4 weeks to induce calli and buds from the calli. Next, culture the calli in a suitable plant regeneration medium for callus (e.g., ECR-3, ECR-2, ECR-6, ECR-7, ECR-8 and ECR-9) at 117.7 μmol m⁻² s⁻¹ and 24° C. under 16 h photoperiod for approximately 3 to 4 weeks for shoot regeneration. Then, culture the multiple shoots/buds in MS basal medium without plant growth hormone at 117.7 μmol m⁻² s⁻¹ and 24° C. under 16 h photoperiod for 1-2 weeks to generate roots for plant regeneration.

Specific examples of methods for regenerating wheat and corn are also provided herein. For example, a method for regenerating wheat plantlets from calli can include the following: sterilizing and germinating wheat seeds resulting in germinated seedlings; isolating leaf explants from the germinated seedlings under sterile conditions; culturing the leaf explants in embryonic callus induction medium under sterile conditions such that calli are produced and bud primordia grow from the calli; culturing the calli in plant regeneration medium for callus under sterile conditions such that shoots grow from the calli; and culturing the shoots and/or buds under conditions such that roots grow from the shoots resulting in production of wheat plantlets. The method can further include subjecting the wheat plantlets to chemical mutagenesis. In one particular embodiment of this method, the following steps are followed. First, sterilize and germinate seeds using the protocol set forth in Example 9. Second, isolate leaf explants (approximately 0.2 to 0.3 cm in length from the base of leaf) from germinated seedlings. Next, culture leaf explants in a suitable embryonic callus induction medium (e.g., EC-8, EC-9 and EC-11) in the dark at 26° C. for approximately 4 weeks to induce calli and buds from the calli. Next, culture the calli in a suitable plant regeneration medium for callus (e.g., ECR-3, ECR-8 and ECR-9) at 117.7 μmol m⁻² s⁻¹ and 24° C. under a 16 h photoperiod for approximately 4 to 8 weeks for shoot regeneration. Then, culture the multiple shoots/buds in new plant regeneration medium for callus (e.g., ECR-3) or a suitable multiple shoot induction medium (e.g., MI-2 medium) at 117.7 μmol m⁻² s⁻¹ and 24° C. under 16 h photoperiod for 3 to 4 weeks for root growth and plant regeneration.

Similarly, a method for regenerating corn plantlets and plants from calli can include the following: isolating explants under sterile conditions; culturing the explants in embryonic callus induction medium for callus under sterile conditions such that calli are produced and buds grow from the calli; culturing the calli in plant regeneration medium for callus under conditions such that shoots grow from the calli; and culturing the shoots and/or buds under conditions such that roots grow from the shoots resulting in production of corn plantlets. Typically, the explants are composite meristem explants from at least one of in vitro corn plant culture and leaf explants from corn plant seedlings. The method can further include subjecting the corn plantlets to chemical mutagenesis. In one particular embodiment of this method, the following steps are followed. First, isolate composite meristem explants from an in vitro plant culture and/or leaf explants (approximately 0.5 cm in length from the base of leaf) from seedlings. Second, culture the explants in a suitable embryonic callus induction medium (e.g., EC-8 and EC-11) in the dark at 26° C. for approximately 4 weeks to induce calli and buds from the calli. Next, culture the calli in a suitable plant regeneration medium for callus (e.g., ECR-3, ECR-8 and ECR-9) at 117.7 μmol m⁻² s⁻¹ and 24° C. under a 16 h photoperiod for approximately 4 to 8 weeks for shoot regeneration. Next, culture the multiple shoots/buds in a suitable plant regeneration medium for callus (e.g., ECR-3) or a suitable multiple shoot induction medium (e.g., MI-2 medium) at 117.7 μmol m⁻² s⁻¹ and 24° C. under a 16 h photoperiod for approximately 3 to 4 weeks for root growth and plant regeneration.

### Media Formulations

Described herein are novel media formulations for large-scale micropropagation and regeneration of a graminaceous plant such as Arundo, wheat or corn (maize). Exemplary embodiments of these media formulations are shown in Table 12. Each of the novel media formulations listed in Table 12 (except where noted) was made using MS basal medium including 4.4 g MS medium with vitamins (commercially available from Phyto Technology Laboratories, Shawnee Mission, Kans.—see Murashige T and Skoog F, Physiol Plant 15: 473-497, 1962), 30 g sucrose (commercially available from VWR), 6.5 to 7 g agar (commercially available from Sigma Aldrich), pH 5.8. In media formulations A4, EC-6, EC-7, EC-8, EC-9, EC-10 and EC-11, however, 1.95 g MES hydrate (commercially available from Sigma Aldrich) was added. Although the experiments described herein involved the use of MS basal medium, any suitable basal medium can be used. A novel media formulation may include a MS basal medium which itself includes or to which has been added sucrose and/or vitamins.

Each multiple shoot induction medium (also referred to herein as A2, MI-2, MI-3, MI-4, and MI-5) also contains one or more auxins and one or more cytokinins In a multiple shoot induction medium formulation, an auxin at a concentration of 0.1 to 4 mg/l (e.g., 0.1, 0.5, 0.7, 0.9, 1.0, 1.5, 2.0, 2.5, 3.0, 3.5, 4.0, etc.) is typically included. Examples of auxins include 2,4-D, Dicamba, Pichloram, IBA, NAA and/or IAA. If 2,4-D is used, it is generally used at a concentration of 0.5 to 4 mg/l (e.g., 0.5, 0.7, 0.9, 1.0, 1.5, 2.0, 2.5, 3.0, 3.5, 4.0, etc.). If IBA is the auxin added to the medium, IBA is typically added at a concentration of 0.1 to 2 mg/l (e.g., 0.1, 0.5, 0.7, 0.9, 1.0, 1.5, 2.0, etc.). A cytokinin can be added at a concentration of 0.05 to 5 mg/l (e.g., 0.05, 0.1, 0.5, 1.0, 1.5, 2.0, 2.5, 3.0, 3.5, 4.0, 4.5, 5.0, etc.). Examples of cytokinins include BA, and thidiazuron (TDZ) as well as kinetin, zeatin, and isopentenyl adenine (2ip).

A typical multiple shoot induction medium includes:
0.5 to 4 mg/l of a first auxin (e.g., 2,4D), and 0.1 to 5 mg/l of a first cytokinin (e.g., BA); and
optionally, one or more of:
0.1 to 2 mg/l of a second auxin (e.g., IBA), and 0.05 to 2.5 mg/l of a second cytokinin (e.g. TDZ).

The root induction medium formulations of Table 12 also include MS basal medium supplemented with vitamins and sucrose (e.g., 30 g/l sucrose). Root induction medium MGM_{—}1 of Table 12 also includes 2.2 g/l MS Basal salt mixture and 1.0 mg/l IBA.

A typical root induction medium comprises:
2.2 to 4.4 g/l of Basal salt mixture; and
0.5 to 2 mg/l IBA.

Another typical root induction medium comprises MS basal medium supplemented with vitamins, sucrose, and agar, having a pH of approximately 5.8.

The callus induction medium formulations shown in Table 12 are made with MS basal medium supplemented with vitamins and sucrose (e.g., 30 g/l sucrose). Use of callus induction medium covers embryonic and organogenic callus formation which are two pathways for plant regeneration. A typical callus induction medium also includes MES hydrate (e.g., 1.95 g MES hydrate) and an auxin (e.g., 2,4D). An callus induction medium can further include one or more of: a cytokinin. In an callus induction medium, an auxin (e.g., 2,4D) at a concentration of 0.01 to 2 mg/l (e.g., 0.01, 0.05, 0.1, 0.5, 1.0, 1.5, 2.0, etc.) is typically included. A cytokinin (e.g., BA) can be added at a concentration of 0.25 to 4 mg/l (e.g., 0.25, 0.5, 1.0, 1.5, 2.0, 2.5, 3.0, 3.5, 4.0, etc.).

A typical callus induction medium comprises:
1 to 4 mg/l of an auxin;
1.95 g MES hydrate; and
optionally, a cytokinin at a concentration of 0.1 to 2 mg/l.

As shown in Table 12, plant regeneration medium for callus formulations are made with basal MS medium supplemented with vitamins and sucrose (e.g., 30 g/l sucrose). A typical plant regeneration medium for callus formulation also includes a cytokinin (e.g., BA). A cytokinin can be added at a concentration of about 0.1 mg/l (e.g., 0.09, 0.1, 0.15, etc.). A plant regeneration medium for callus formulation can further include an auxin. If 2,4D is included as an auxin, it is generally present at a concentration of 2 mg/l (e.g., 1.9, 2.0, 2.1, etc.).

A typical plant regeneration medium for callus formulation comprises one or more of:
0.25 to 4 mg/l of a cytokinin, and optionally:
0.01 to 2 mg/l of an auxin.

Examples of suitable plant regeneration medium for callus formulations are listed in Table 12, and include ECR-2, ECR-3, ECR-5, ECR-6, ECR-7, ECR-8 and ECR-9.

### Systems for Micropropagating and Regenerating Graminaceous Plants and Plantlets

A system for micropropagating graminaceous plants and plantlets is described herein and includes: at least one sample of graminaceous composite meristem explant isolated from plant seedling or in vitro plantlets for use as an explant to induce shoot induction and root growth; multiple shoot induction medium; basal medium lacking plant growth hormones; and root induction medium. In a specific embodiment of a system for micropropagating wheat plantlets on a large scale, the system can include at least one sample of wheat composite meristem explant isolated from plant seedlings or in vitro plantlets for use as an explant to induce shoot induction and root growth; multiple shoot induction medium; and basal medium including sucrose, vitamins and a cytokinin. In this specific embodiment, the system can also include reagents and equipment or devices for sterilizing and germinating wheat seeds for generating germinated wheat seedlings. A system for micropropagating graminaceous plants can also include one or more Peat plugs and/or a vacuum system for culturing the composite meristem explants and the shoots and/or buds. In a system, one or more of the media may be liquid and/or solid media. The system can be used to propagate any graminaceous grass with modifications to the conditions and media formulations described herein. The system can be used for large-scale propagation of graminaceous plants. Examples of graminaceous plants that can be propagated on a large scale include Arundo and its transgenic plants, corn, wheat, and other grass plants.

Systems for regenerating a graminaceous plantlet from a callus are also described herein. In one embodiment, a system for regenerating Arundo plantlets (and plants) includes at least one sample of composite meristem explant, root explant, or leaf explant for use as an explant to induce shoot induction and root growth; embryonic and/or organogenic callus induction medium; plant regeneration medium for callus; and basal medium lacking plant growth hormones. In another embodiment, a system for regenerating wheat plantlets (and plants) includes: at least one sample of composite meristem explant and/or leaf explants from germinated wheat seedlings to induce shoot induction and root growth; embryonic and/or organogenic callus induction medium; and plant regeneration medium for callus. In yet another embodiment, a system for regenerating corn plantlets (and plants) includes at least one sample of leaf explants and/or composite meristem explants from in vitro corn plant culture or from corn plant seedlings to induce shoot induction and root growth; embryonic callus induction medium for callus; and plant regeneration medium for callus.

Such systems can be packaged as a kit for commercial use. A kit for micropropagating graminaceous plants and/or regenerating graminaceous plants via callus phase would typically include instructions for use and appropriate packaging. A kit for regenerating graminaceous plants may also include reagents and instructions for subjecting the plants (plantlets) to chemical mutagenesis.

### EXAMPLES

The present invention is further illustrated by the following specific examples. The examples are provided for illustration only and should not be construed as limiting the scope of the invention in any way.

### Example 1

### A Novel Micropropagation and Plant Regeneration System via Callus Phase for Arundo donax and Other Monocots

Giant reed (Arundo donax L.) is exclusively and vegetatively-propagated from fragments of stems and rhizomes due to its reproductive sterility, which is time-consuming and cost expensive, and limits large-scale cultivation. Therefore, to alleviate these negative aspects, we have developed: 1) a high-throughput micropropagation system with up to a 30-fold shoot multiplication rate and 100% rooting efficiency, using in vitro established shoot/bud cultures as explants; and 2) an efficient plant regeneration system via a callus phase from composite meristem explant, and leaf explants, with up to 164 shoots generated from a single callus unit (approximately 0.5 cm in diameter), with a 100% rooting efficiency.

The following five new methods described below were developed to address the production problem and promote breeding programs for Arundo, as well as other monocot species.

I. Development of High Throughput Micropropagation Systems for Arundo donax and Other Monocots.

These systems included two new technologies described below.

1. Development of Regeneration System Using Composite Meristem Explant Isolated From In Vitro Plants as Explant

By investigating different plant growth regulators, in combination with culture media and composite meristem explants containing root, apical and intercalary meristems as explants, a high throughput system has been developed for multiple shoot induction and rooting for Arundo genotypes, including genotype CMT1 and two commercial cultivars of Arundo donax, cvs. Peppermint Stick and Variegata. Using this system, 10- to 36-fold shoot production rates were obtained within 3 to 4 weeks. One hundred percent of the shoots produced roots within 2 to 3 weeks. Using this method, massive shoot production (up to more than 60 shoots/buds) was obtained from a single composite meristem explants. This new technology shows 3.6 to 14.4-fold greater propagation efficiencies compared to all previous methods described, in terms of number of plants produced, and time frame for plant propagation.

A typical high throughput regeneration system has the following characteristics: 1) large scale production capability, 2) rapid production cycle, which takes, for example, 4 to 6 weeks from explants to plants which are ready to soil, 3) minimal production complexity, which had a simple protocol with one to two single steps (plant regeneration in one step or shoot induction and rooting in two steps), 4) inexpensive methodology and 5) high quality of regenerated plants without off-type plants.

2. Develop High Throughput Production System and Produce Arundo Plants

A high throughput system has been developed for large scale Arundo production. This system includes: 1) production culture procedure; and 2) a device for efficiently performing culture transfer. For the production culture procedure, explant unit containing 2-4 composite meristem explants or 2-4 shoots and/or buds were cultured in baby jars (subsequently placed in plastic boxes for the culture period) using medium MI-2 for 2-4 weeks, to produce multiple shoots. These multiple shoots/buds were then cultured in MS basal medium without plant growth hormone for 1-2 weeks to generate roots. Following root production, the plants were transferred into Peat Plugs containing 50 ml of liquid medium MGM_{—}1 without sugar, under non-sterile conditions for 2 weeks. MGM_{—}1 medium without sugar enabled Arundo plants generated from tissue culture to grow in the Peat Plugs for approximately 1-2 months under non-sterile conditions. In addition the preparation of MGM_{—}1 medium and transfer of the plants from tissue culture into Peat Plugs were under non-sterile condition, which simplified medium preparation and culture transfer process. Two weeks after culture in Peat Plugs, the plugs with the plants were transplanted in the field. For the very first delivery, almost 100% of the plants survived in the field after transplanting. The device developed for the efficient culture transfer is composed of: 1) a vacuum system and 2) a sterile pipetting tool. This device allowed culture transfers approximately 3 times faster than by hand transfer.

### II. Develop Efficient Plant Regeneration System via Callus Phase Using Composite Meristem, Root and Leaf Explants for Arundo

This system included three new technologies described below.

1. Development of Efficient Regeneration System via Callus Phase Using Composite Meristem Explants

Efficient plant regeneration systems via callus phase have been developed using composite meristem explant. Approximately 66% of the explants produced organogenic calli from 4-6 weeks after culture. Among the calli produced, 92.9% of them generated shoots and/or buds and 100% of the shoots generated roots 1-2 weeks after culture. In addition, 100% of calli derived from the composite meristem explant generated embryogenic and/or organogenic calli and 75% of them produced bud primordia or embryos. Using this system, we are able to produce up to approximately 164 shoots/buds from a single callus unit. This new technology is 16- to 64-fold more efficient than all previous methods described in terms of plant propagation numbers.

2. Development of Efficient Regeneration System via Callus Phase Using Root Explants

Using this callus system with root explants, 100% of explants produced embryogenic like-calli for tested genotypes CMT1 and Arundo donax cv. Variegata. To induce embryogenic callus and regeneration, four additional media were tested, and up to 100% of explants generated embryogenic calli. In addition, bud primordia and embryo structures were obtained from the calli derived from root explants.

3. Development of Efficient Regeneration System via Callus Phase Using Leaf Explants

The similar combinations of plant growth hormones used for root explants were investigated using leaf explants. Up to 91.7% of leaf explants produced organogenic calli and 100% of explants generated embryogenic calli for genotype CMT1. Bud primordia and embryo structures were obtained from the calli derived from leaf explants. Using this system, up to approximately 210 plants, shoots and/or buds were produced from a single callus unit. This new technology is 16- to 64-fold more efficient than the previous methods described in terms of plant propagation numbers in Arundo donax.

These novel high throughput microppropagation systems and regeneration systems via callus phase will efficiently enable Arundo production and facilitate breeding programs for other monocot species including corn and wheat.

I. Development of High Throughput Micropropagation Systems for Arundo donax

### 1. Development of Regeneration System Using Composite Meristem Explants

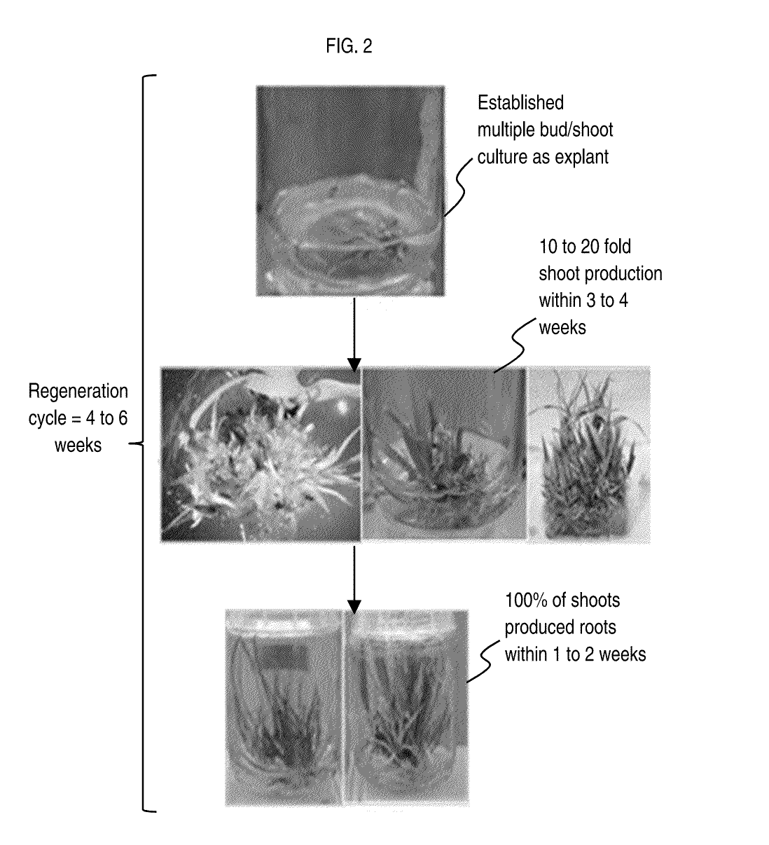

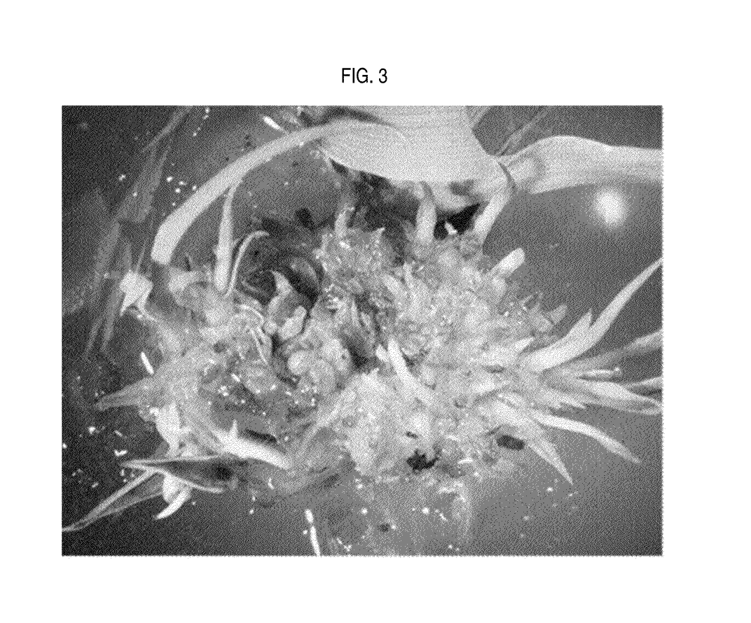

By investigating different plant growth regulators and their combination with culture media, a high throughput system for multiple shoot induction and rooting for Arundo genotypes, including genotype CMT1 and two commercial cultivars of Arundo donax (cvs. Peppermint Stick and Variegata) were developed using composite meristem explants explants. Using this system, 10- to 36-fold shoot production rates were obtained within 3 to 4 weeks for the three genotypes tested. One hundred percent of shoots produced roots within 2 to 3 weeks (Table 1, Table 2 and FIG. 2). Using this method, massive shoot production (up to more than 60 shoots/buds) was obtained from a single shoot for the three genotypes (FIG. 3).

TABLE 1
Multiple shoot production using composite meristem explants as explant
for Arundo genotype CMT1 approximately 3 to 4 weeks after culture.
Stock shoot cultures were maintained in MI-2 medium.

| Treatment | No. explants | Mean No. shoots produced per explant |
|---|---|---|
| MI-4 | 8 | 21.0 distinct shoots (plus 10-30 small buds) |
| MI-3 | 8 | 36.0 distinct shoots (plus 10-30 small buds) |
| MI-2 | 4 | 24.8 distinct shoots (plus 10-30 small buds) |
| MI-5 | 40 | 18.5 |

TABLE 2
Root production from shoots 2 to 3 weeks after culture for Arundo
genotype CMT1.

| Treatment | No. explants | % of shoots producing roots |
|---|---|---|
| MS basal medium | 27 | 100% |

In summary, by using this meristem system, a 10- to 36-fold increase in the number of shoots over a 3 to 4 week period was obtained, with 100% of these shoots rooting in 2 to 3 weeks, for the Arundo genotypes tested.

### Example 2

### Develop High Throughput Production System and Produce Arundo Plants

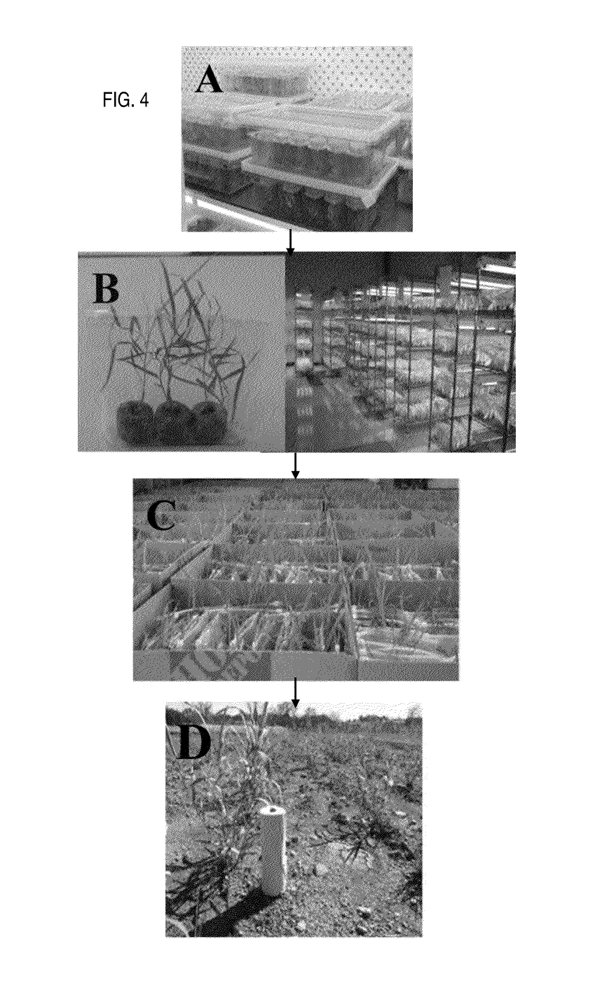

A high throughput production system for large scale Arundo production (FIG. 4) has been developed. This system includes: 1) production culture procedure (FIG. 4); and 2) a device for efficiently performing culture media transfer (FIG. 5). For the production culture procedure, each explant unit containing 2-4 composite meristem explants or 2-4 shoots and/or buds was cultured in baby jars using medium MI-2 (that were placed in plastic boxes) for 2 to 4 weeks to produce multiple shoots and the multiple shoots/buds were cultured in MS basal medium without plant growth hormone for 1-2 weeks to generate roots (FIG. 4 panel A). Following root production, the plants were transferred into Peat Plugs containing 50 ml of liquid medium MGM_{—}1 without sugar under non-sterile conditions for 2 weeks (FIG. 4 panel B). MGM_{—}1 medium without sugar enabled Arundo plants generated from tissue culture to grow in the Peat Plugs for approximately 1 to 2 months under non-sterile condition. In addition the preparation of MGM_{—}1 medium and transfer of the plants from tissue culture into Peat Plugs were under non-sterile condition, which simplified medium preparation and culture transfer process. Two weeks after culture in Peat Plugs, the Peat Plugs with the plants were transplanted in the field (FIG. 4 panel D). For the very first delivery almost 100% of the plants survived in the field after transplanting (FIG. 4 panel D).

For the device for efficiently performing culture medium transfer, the device is composed of: 1) a vacuum system 10; and 2) medium transfer tool 20 comprising a sterile pipetting tool (FIG. 5). This device transferred culture medium 3 times faster than hand transfer (an average of 2 min with the device vs. 6 min 11 seconds with hand for transferring 10 baby jar culture), significantly speeding up the subculture process.

### Example 3

### Develop Plant Regeneration System via Callus Phase Using Composite Meristem Explant, Root and Leaf as Explants

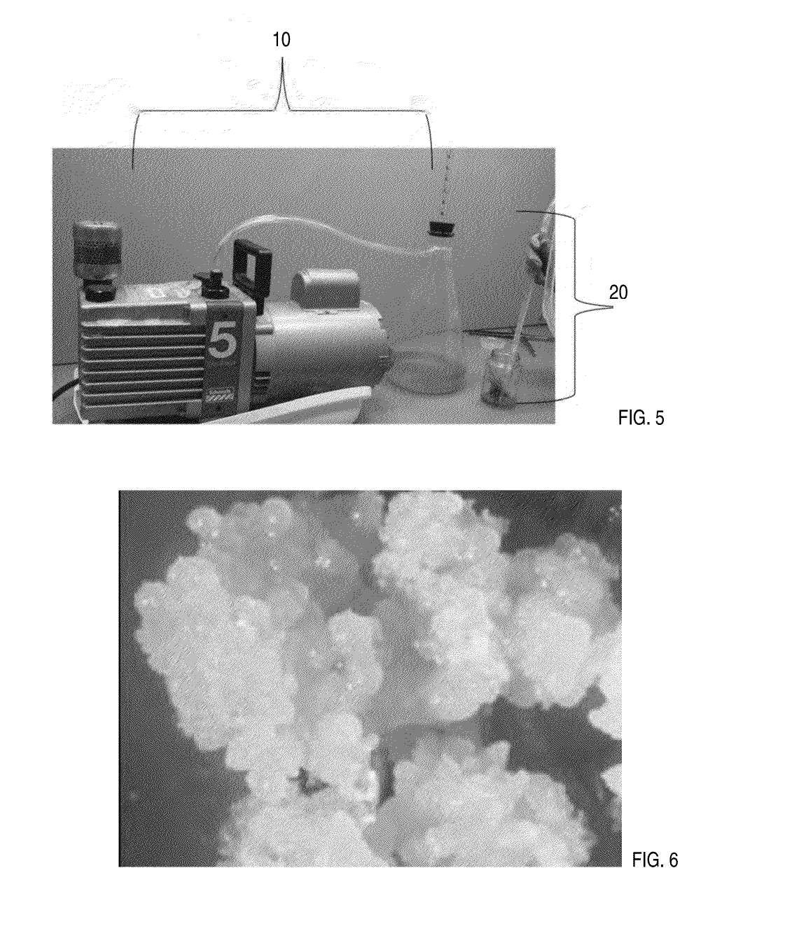

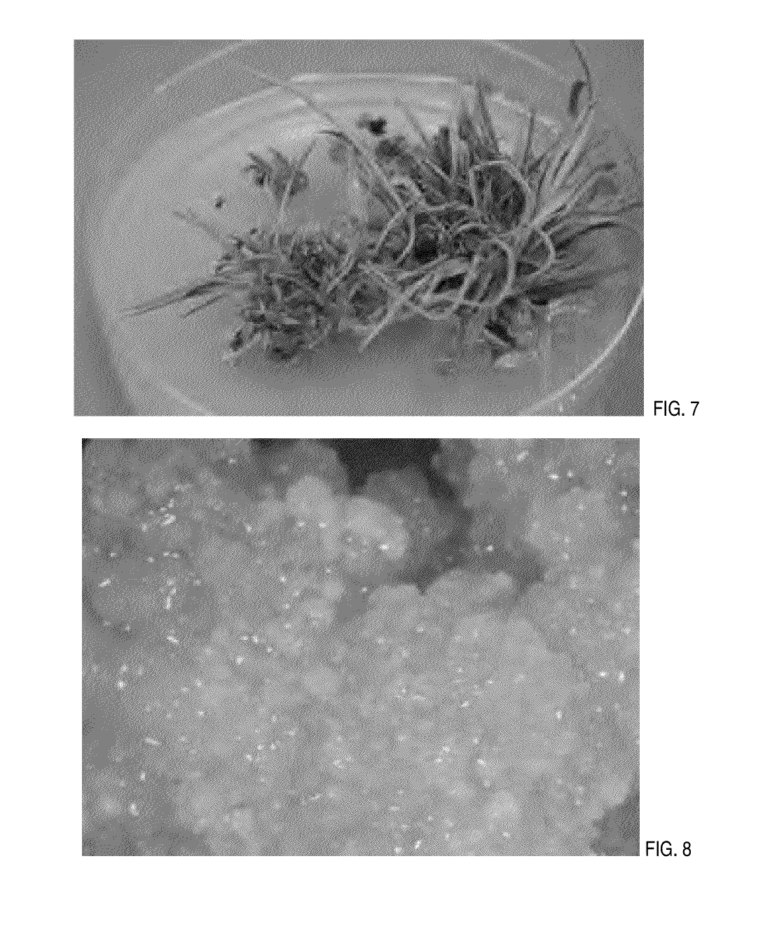

Develop plant regeneration system via callus phase using composite meristem explant: Based on the results of initial experiments for callus induction, two optimal media were selected for callus induction and tested using composite meristem explants. An efficient plant regeneration system via callus phase using composite meristem explants. Approximately 66% of explants produced organogenic calli (Table 3 and FIG. 6) 4 to 6 weeks after culture. Among the calli produced, 92.9% of them generated shoots and/or buds up to 164 shoots/buds obtained from single callus unit (Table 4 and FIG. 7), 100% of the shoots generated above produced roots 1 to 2 weeks after culture (Table 5). This regeneration system via callus phase can be used for Arundo chemical mutagenesis breeding. In addition 100% of calli derived from the composite meristem explants generated embryogenic-like calli and 75% of them produced bud primordia or embryos in medium EC-11 (Table 6 and FIG. 8).

TABLE 3
Organogenic callus induction from composite meristem explants
for genotype CMT1.

| | | Percentage of explants producing |
|---|---|---|
| Medium | No. of explant | organogenic calli with buds |
| EC-6 | 18 | 66.7 |
| A4 | 18 | 33.3 |

TABLE 4
Shoot regeneration from the calli derived from composite meristem
explants for genotype CMT1.

| | No. of | Percentage of calli producing |
|---|---|---|
| Medium | explant | shoots and buds |
| ECR-2/calli from medium EC-6 | 14 | 92.9 |
| ECR-2/calli from medium A4 | 12 | 16.7 |

TABLE 5
Plant regeneration from the shoots regenerated from the calli derived
from composite meristem explants for genotype CMT1.

| Medium | No. of explant | Percentage of shoots producing roots |
|---|---|---|
| MS basal medium | 40 | 100 |
| without hormone | | |

TABLE 6
Embryogenic-like callus induction from composite meristem explants
of genotype CMT1.

| | | Percentage of explants | Percentage of explants |
|---|---|---|---|
| | No. of | producing embryogenic- | producing |
| Medium | explants | like calli | bud primordial or embryos |
| EC-6 | 36 | 100 | 8.3 |
| EC-7 | 24 | 100 | 20.8 |
| EC-8 | 24 | 100 | 29.1 |
| EC-9 | 24 | 100 | 29.1 |
| EC-10 | 24 | 100 | 37.5 |
| EC-11 | 24 | 100 | 75.0 |

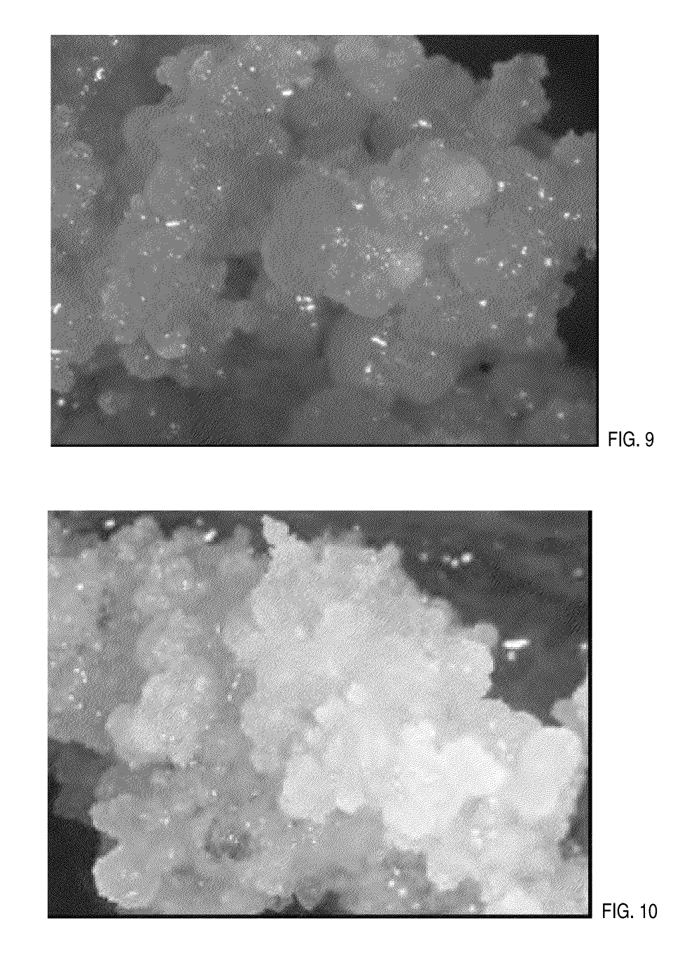

Develop plant regeneration system via callus phase using root explants for Arundo donax: By first investigating root explants (root tip approximately 1 cm long) and plant growth regulators, 61.9% of root explants produced calli in medium A4. Four weeks after the culture, the calli were transferred into different media (Tables 7, 8 and 9) to induce embryogenic-like calli and regeneration. Four weeks after the culture, 100% of explants produced embryogenic like-calli each for genotype CMT1 and Arundo donax cv. Variegata (Tables 7 and 8). To induce embryogenic callus and regeneration, four additional media were tested and up to 100% of explants generated embryogenic calli (Table 9 and FIG. 9). In addition bud primordia and embryo-like structure were obtained from the calli derived from root explants (FIG. 10).

TABLE 7
Embryogenic-like callus induction from root explants of Arundo donax
cv. Variegata.

| | | Percentage of explants producing |
|---|---|---|
| Medium | No. of explant | embryogenic-like calli |
| A4 | 36 | 0 |
| EC-6 | 24 | 100 |
| EC-1 | 36 | 81.3 |
| EC-7 | 24 | 100 |

TABLE 8
Embryogenic-like callus induction from root explants of genotype CMT1.

| | | Percentage of explants producing |
|---|---|---|
| Medium | No. of explant | embryogenic-like calli |
| EC-1 | 24 | 45.8 |
| EC-6 | 12 | 100 |
| EC-7 | 12 | 100 |

TABLE 9
Embryogenic-like callus growth from root explants of genotype CMT1.

| | | Percentage of explants | Percentage of the |
|---|---|---|---|
| | No. of | producing embryogenic- | embryogenic-like calli with |
| Medium | explants | like calli | 1 to 2 cm in diameter |
| EC-6 | 33 | 97.0 | 33.3 |
| EC-7 | 39 | 97.4 | 38.5 |
| EC-8 | 35 | 100 | 60.0 |
| EC-9 | 39 | 100 | 51.3 |
| EC-10 | 35 | 100 | 34.3 |
| EC-11 | 26 | 100 | 50.0 |

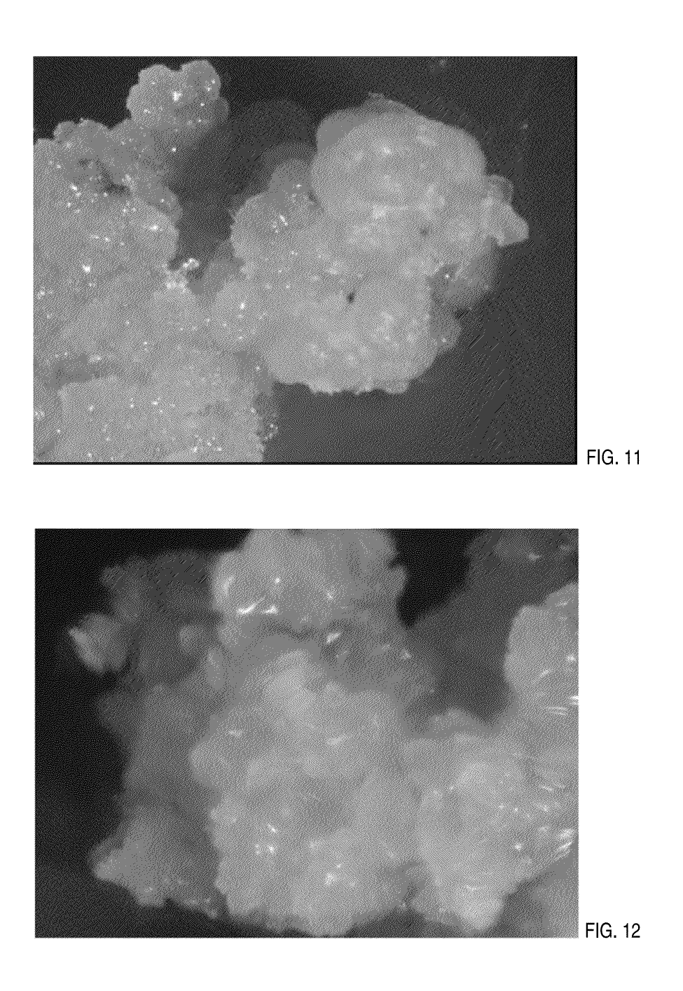

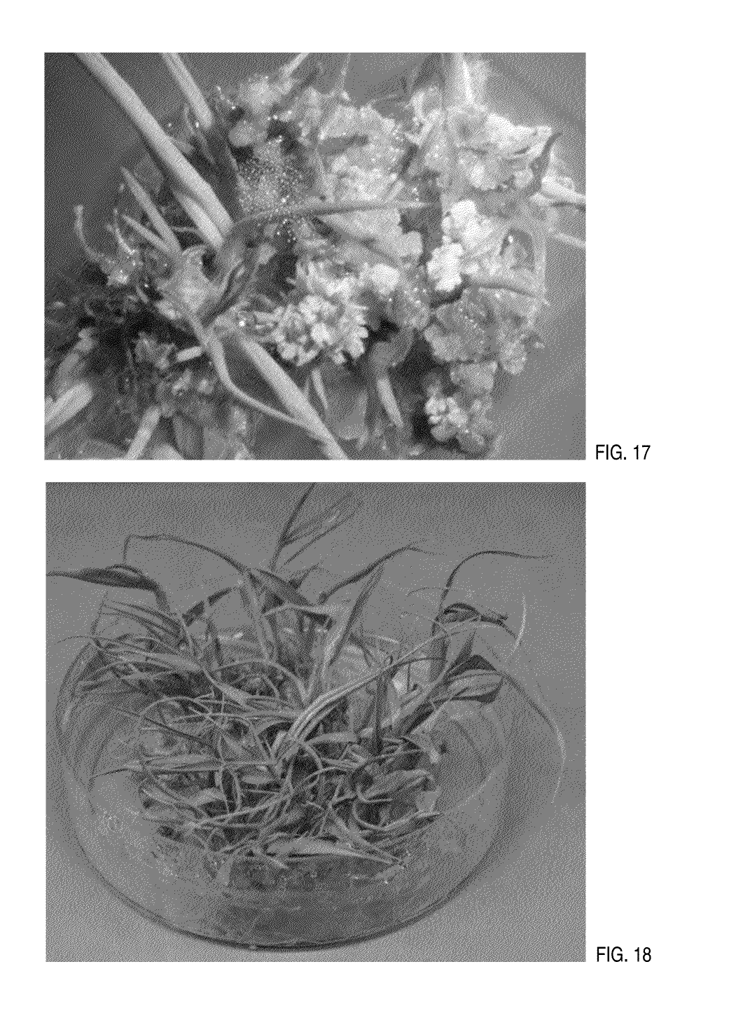

Develop plant regeneration system via callus phase using leaf as explants for Arundo donax: The similar combinations of plant growth hormones were investigated using leaf explants. Up to 91.7% of explants produced organogenic calli (Table 10) and 100% of explants generated embryogenic-like calli (Table 11) for genotype CMT1. Bud primordia (FIG. 11) and embryo-like structures (FIG. 12) were obtained from the calli derived from leaf explants. Approximately up to 210 plants, shoots and/or buds were regenerated from a single callus unit (FIGS. 17 and 18).

TABLE 10
Organogenic callus induction from leaf explants of genotype CMT1.

| | | Percentage of explants |
|---|---|---|
| Treatment | No. of explant | producing organogenic calli |
| A4 | 48 | 12.5 |
| EC-1 | 12 | 58.3 |
| EC-6 | 12 | 58.3 |
| EC-7 | 12 | 91.7 |

TABLE 11
Embryogenic-like callus induction from leaf explants of genotype CMT1.

| | | Percentage of explants | Percentage of the |
|---|---|---|---|
| | No. of | producing embryogenic- | embryogenic-like calli with |
| Medium | explants | like calli | 1 to 2 cm in diameter) |
| EC-6 | 12 | 41.7 | 0 |
| EC-7 | 12 | 75.0 | 0 |
| EC-8 | 30 | 83.3 | 46.7 |
| EC-9 | 12 | 100 | 41.7 |
| EC-10 | 20 | 95 | 35.0 |
| EC-11 | 22 | 100 | 18.2 |

### Example 4

### Media Formulations

All media were formulated based on MS basal medium included 4.4 grams (g) MS medium with vitamins (Product No: M519, Phyto Technology Laboratories), 30 g sucrose (Product No: BDH0308, VWR), 6.5 to 7 g agar (Prod. No: A7921, Sigma Aldrich), pH: 5.8, except 1.95 g MES hydrate (M2933-25g, Sigma) were added to media A4, EC-6, EC-7, EC-8, EC-9, EC-10 and EC-11.

TABLE 12
Media Formulations

| Name of medium | Plant growth hormones |
|---|---|
| Multiple shoot induction medium | |
| MI-2 | 2.5 mg/l BA, 0.2 mg/l 2,4D |
| MI-3 | 3 mg/l BA, 0.05 mg/l IBA, |
| | 0.05 mg/l 2,4D |
| MI-4 | 3 mg/l BA, 0.05 mg/l TDZ, |
| | 0.05 mg/l 2,4D |
| MI-5 | 5 mg/l BA, 0.2 mg/l 2,4-D |
| A2 | 2.5 mg/l TDZ, 0.5 mg/l 2,4-D |
| Root induction medium | |
| MS basal medium | |
| MGM_1 | 2.2 g/l MS Basal salt mixture, |
| | 1 mg/l IBA |
| Embryonic callus induction medium | |
| A4 | 4 mg/l 2,4D, 1 mg/l BA |
| EC-1 | 4 mg/l 2,4D, 2 mg/l BA |
| EC-6 | 4 mg/l 2,4-D, 1 mg/l BA, |
| | 10 mg/l lipoic acid |
| EC-7 | 4 mg/l 2,4-D, 1 mg/l BA, |
| | 10 mg/l melatonin |
| EC-8 | 3 mg/l 2,4 D |
| EC-9 | 3 mg/l 2,4 D, 0.1 mg/l BA |
| EC-10 | 4 mg/l 2,4 D, 0.5 mg/l BA |
| EC-11 | 2 mg/l 2,4 D |
| Plant regeneration medium for callus | |
| MS basal medium | |
| ECR-2 | 2 mg/l BA, 0.01 mg/l 2,4-D |
| ECR-3 | 0.5 mg/l BA |
| ECR-5 | 1 mg/l BA |
| ECR-6 | 0.25 mg/l BA, 0.1 mg/l IAA |
| ECR-7 | 4 mg/l Kinetin, 1 mg/l IAA |
| ECR-8 | 1 mg/l Kinetin, 0.5 mg/l IAA |
| ECR-9 | 0.5 mg/l BA, 0.1 mg/l NAA |

### Example 5

### Apply Arundo Regeneration Methods to Corn, Genotype H99 for Plant Regeneration via Callus Phase

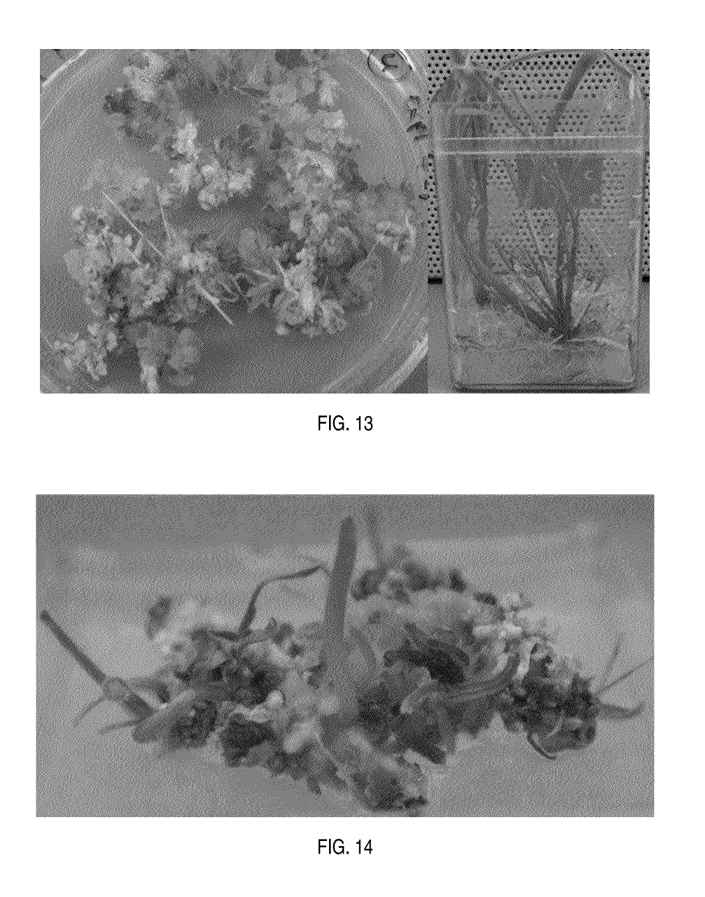

Arundo regeneration methods using composite meristem and leaf as explants were adapted to corn genotype H99. Two optimal Arundo media EC-8 and EC-11, and both composite meristem and leaf explants were used for corn plant regeneration via callus phase. Up to 55.0% of composite meristem explants produced on organogenic or embryonic-like calli approximately 4 weeks after culture and 54.6% of the explants generated buds or shoots 4 weeks after culture in medium EC-11 (Tables 13 and 14). Plant regeneration was obtained using the composite meristem explants (FIG. 13). For leaf explant, 17.1% of explants regenerated buds and shoots 4 to 8 weeks after culture in medium EC-8 (Table 15) and plant regeneration was obtained (FIG. 14).

### Example 6

### Apply Arundo Regeneration Methods to Wheat, Genotype Bobwhite for Plant Regeneration with and without Callus Phase

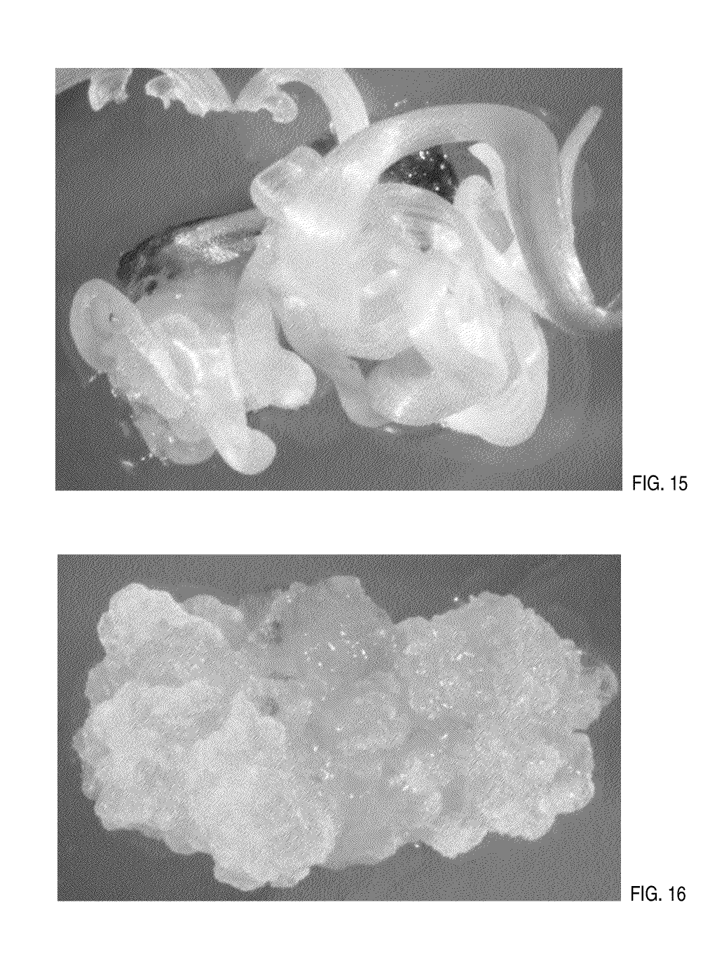

Arundo regeneration methods using composite meristem and leaf as explants were also adapted to wheat genotype Bobwhite. Two optimal Arundo media A2 and MI-2 and composite meristem explants were used for direct plant regeneration without callus phase. Also, two optimal media EC-8 and EC-11 and leaf explants were used for plant regeneration via callus phase. For composite meristem explant, 38.5% of the explants generated multiple buds or shoots without callus phase 4 weeks after culture in A2 medium (Table 16, and FIG. 15). For leaf explant, 45.3% of the explants produced calli 4 weeks after culture in EC-8 medium, and 70% of explants generated calli and 20% of the explants formed bud primordial 8 weeks after culture in EC-8 and EC-11 media. Media EC-8 and EC-11 were optimal for callus induction and bud formation compared to other medium EC-9 (Tables 17 and 18 and FIG. 16).

In summary, Arundo medium EC-11 was effective for organogenic or embryonic-like callus induction and plant regeneration for corn when using composite meristem explant. Arundo EC-8 medium was optimal to regenerated shoots and plants for corn when using leaf explants. For wheat, Arundo medium A2 was more effective for generating multiple buds or shoots than Arundo medium MI-2 when using composite meristem explant. Arundo EC-8 and EC-11 media were optimal for inducing calli and bud primordium compared with medium EC-9 when using leaf explant. All Arundo media tested in corn and wheat using composite meristem and leaf explants successfully regenerated bud primordia, buds and/or plants, indicating Arundo regeneration systems using both composite meristem and leaf explants were less genotype-dependent and can be used for corn and wheat plant regeneration with or without callus phase.

TABLE 13
Effect of different Arundo media on organogenic or embryonic-like callus
induction using composite meristem explants in corn approximately
4 weeks after culture (H99).

| | | % of explants | | | | | |
|---|---|---|---|---|---|---|---|
| | | producing | | | | | |
| | | organogenic | | | | | |
| Treat- | No. of | or embryonic- | % of calli in different size^{x} | | | | |
| ment | explants | like calli | + | ++ | +++ | ++++ | +++++ |
| EC-8 | 18 | 33.3 | 0 | 5.6 | 16.7 | 5.6 | 5.6 |
| EC-11 | 60 | 55.0 | 10.0 | 13.3 | 6.7 | 15.0 | 10.0 |

^{x}+: ≦0.5 cm in diameter; ++: 0.5 cm < and ≦1 cm in diameter; +++: 1 cm < and ≦2 cm in diameter; ++++: 2 cm < and ≦3 cm in diameter; +++++: 3 cm < and ≦4 cm.

TABLE 14
Effect of different Arundo media on bud and shoot regeneration using
composite meristem explant in corn (H99).

| | | % of explants producing |
|---|---|---|
| Treatment | No. of explants | calli generating buds and shoots |
| EC-8 | 19 | 47.4 |
| EC-11 | 44 | 54.6 |

TABLE 15
Effect of different Arundo media on bud and shoot regeneration using leaf
explant in corn (H99).

| | | % of explants having |
|---|---|---|
| Treatment | No. of explants | bud primordial, buds or shoots |
| EC-8 | 41 | 17.1 |
| EC-11 | 20 | 5.0 |

TABLE 16
Effect of different Arundo media on bud and shoot regeneration using
composite meristem explant in wheat (Bobwhite).

| Treatment | No. of Explants | % of explants with multiple buds or shoots |
|---|---|---|
| A2 | 26 | 38.5 |
| MI-2 | 26 | 15.4 |

TABLE 17
Effect of different Arundo media on callus induction using leaf explant
in wheat (Bobwhite) 4 weeks after culture.

| | No. of | % of explants | % of calli in different size^{x} | | | |
|---|---|---|---|---|---|---|
| Treatment | Explants | producing calli | + | ++ | +++ | ++++ |
| EC-8 | 53 | 45.3 | 13.2 | 7.6 | 20.8 | 3.8 |
| EC-11 | 50 | 36.0 | 12.0 | 4.0 | 8.0 | 12.0 |
| EC-9 | 52 | 21.2 | 9.6 | 1.9 | 5.8 | 3.9 |

^{x}+: ≦0.2 cm in diameter; ++: 0.2 cm < and ≦ 0.5 cm in diameter; +++: 0.5 cm < and ≦ 1 cm in diameter; ++++: 1 cm < and ≦ 1.5 cm in diameter.

TABLE 18
Effect of different Arundo media on callus and bud primordium induction
using leaf explant in wheat (Bobwhite) 8 weeks after culture.

| | No. of | % of explants | % of explants with calli |
|---|---|---|---|
| Treatment | Explants | producing calli | producing bud primordia |
| EC-8 | 40 | 70.0 | 20.0 |
| EC-11 | 27 | 70.4 | 22.2 |
| EC-9 | 27 | 25.9 | 11.1 |

### Example 7

### Plant Regeneration Using Leaf Explants in Arundo

As shown in FIG. 17, massive shoot and bud regeneration (approximately 94 buds and shoots) from a single leaf explant of Arundo, Genotype CMT1, was achieved. As shown in FIG. 18, massive plant regeneration (total more than approx. 120 plants, shoot and buds) from a single leaf explant of Arundo, genotype CMT1, was achieved using leaf explant described above.

### Example 8

### Method for Sterilizing and Germinating Corn Seeds

Step 1: the first seed surface sterilization and seed softening:
1. Place 15 seeds in a sterile 50 ml falcon tube.
2. Add approximately 20 ml of 70% ethanol and shake the tube at medium speed for 3 min.
3. Decant the ethanol in the hood.
4. Add approximately 20 ml of 50% bleach solution and shake the tube for 30 min.
5. Decant the bleach in the hood.
6. Rinse the seeds 4 times with sterile DD water (˜30 ml each time).
7. After the last rinse, keep seeds in ˜20 ml sterile water and leave the tube inside the hood for 24 hours.

Step 2: the second seed surface sterilization:
8. After 24 hours, sterilize the softened seeds once with 50% bleach solution for 5 min under shaking
9. Decant the bleach and rinse the seeds 3 times with sterile DD water.
10. Transfer the seeds into solid medium ECR-3 plates (5 seeds per plate) and culture at 25° C.±1° C. in the dark for approximately 2 to 3 weeks.

### Example 9

### Method for Sterilizing and Germinating Wheat Seeds

Wheat seeds were sterilized and germinated as follows:
1. Place approximately 100 seeds in a sterile 50 ml falcon tube.
2. Add approximately 20 to 30 ml of 70% ethanol and hand-shake the tube for 1 min.
3. Decant the ethanol in the hood.
4. Add approximately 20 to 30 ml of 20% bleach solution (5.25% sodium hypochlorite) and shake the tube for 20 min.
5. Decant the bleach in the hood.
6. Rinse the seeds 5 times with sterile DD water (˜30 ml each time).
8. Place seeds on a sterile Petri dish containing 3 sterile filter papers and 10 ml sterile DD water with 0.5 mg/l BA for germination at 25° C.±1° C. in the dark for approximately 2 to 3 weeks.

### Other Embodiments

Any improvement may be made in part or all of the reagents, systems and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. Although the experiments described herein involve micropropagation and regeneration of Arundo, corn and wheat, the micropropagation and regeneration methods and media described herein can be used to propagate additional monocot plants on a large-scale. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method for large-scale production of a graminaceous plant comprising:
a) isolating a plurality of composite meristem explants from at least one of in vitro graminaceous plant culture and seedlings under sterile conditions;
b) culturing the plurality of composite meristem explants in multiple shoot induction medium containing at least one plant growth hormone under sterile conditions such that at least one of multiple shoots and multiple buds grow from each composite meristem explant;
c) culturing the at least one of multiple shoots and multiple buds in basal medium lacking plant growth hormones under sterile conditions such that roots grow from the shoots resulting in a plurality of plantlets; and
d) transferring the plurality of plantlets to a substrate comprising a carbohydrate-free medium that enables growth of the plantlets under non-sterile conditions, and propagating the plantlets.

2. The method of claim 1, wherein the at least one of multiple shoots and multiple buds comprises up to approximately 60 shoots per explant, up to approximately 60 buds per explant, or up to approximately 60 shoots and buds per explant.

3. The method of claim 1, wherein the graminaceous plant is selected from the group consisting of: Arundo, corn and wheat.

4. The method of claim 1, wherein the substrate comprises at least one Peat plug.

5. The method of claim 1, wherein a vacuum system is used for culturing in steps b) and c).

6. The method of claim 1, further comprising step e) of transplanting the plurality of plantlets into soil.

7. The method of claim 6, wherein the method provides a rooting efficiency in the range of about 90% to about 100%.

8. A method for large-scale production of wheat plantlets comprising:
a) isolating a plurality of composite meristem explants from at least one of in vitro plantlet culture and germinated seedlings under sterile conditions;
b) culturing the plurality of composite meristem explants in multiple shoot induction medium containing at least one plant growth hormone under sterile conditions such that at least one of multiple shoots and multiple buds grow from each composite meristem explant; and
c) culturing the at least one of multiple shoots and multiple buds in basal medium comprising sucrose, vitamins and a cytokinin under sterile conditions such that roots grow from the shoots resulting in a plurality of wheat plantlets.

9. The method of claim 8, wherein the method further comprises sterilizing and germinating a plurality of wheat seeds resulting in germinated seedlings.

10. A method for regenerating a graminaceous plantlet from a callus comprising:
a) isolating an explant from at least one of in vitro graminaceous plant culture and seedlings under sterile conditions;
b) culturing the explant in embryonic callus induction medium under sterile conditions such that a callus is produced and buds grow from the callus;
c) culturing the callus in plant regeneration medium for callus under sterile conditions such that multiple shoots grow from the callus; and
d) culturing the shoots and buds in basal medium under sterile conditions such that roots grow from the shoots resulting in production of a plantlet.

11. The method of claim 10, wherein the graminaceous plant is Arundo.

12. The method of claim 10, wherein the explant is selected from the group consisting of:
composite meristem explant, root explant, and leaf explant.

13. The method of claim 10, wherein the method further comprises subjecting the callus and the plantlet to chemical mutagenesis and genetic modification via Agrobacterium-mediated and biolistic transformation.

14. A method for regenerating wheat plantlets from calli comprising:
a) sterilizing and germinating a plurality of wheat seeds resulting in a plurality of germinated seedlings;
b) isolating leaf explants from the germinated seedlings under sterile conditions;
c) culturing the leaf explants in embryonic callus induction medium under sterile conditions such that calli are produced and buds grow from the calli;
d) culturing the calli in plant regeneration medium for callus under sterile conditions such that shoots grow from the calli; and
e) culturing the shoots and buds under conditions such that roots grow from the shoots resulting in production of wheat plantlets.

15. The method of claim 14, wherein the method further comprises subjecting the calli and the wheat plantlets to chemical mutagenesis and genetic modification via Agrobacterium-mediated and biolistic transformation.

16. A method for regenerating corn plantlets from calli comprising:
a) isolating explants under sterile conditions, wherein the explants are at least one of composite meristem explants and leaf explants from at least one of: in vitro corn plant culture and seedlings;
b) culturing the explants in embryonic callus induction medium for callus under sterile conditions such that calli are produced and buds grow from the calli;
c) culturing the calli in plant regeneration medium for callus under conditions such that shoots grow from the calli; and
d) culturing the shoots and buds under conditions such that roots grow from the shoots resulting in production of corn plantlets.

17. The method of claim 16, wherein the method further comprises subjecting the corn plantlets to chemical mutagenesis and genetic modification via Agrobacterium-mediated and biolistic transformation.

18. A plurality of graminaceous plantlets produced according to the method of claim 1.

19. A plurality of wheat plantlets produced according to the method of claim 8.

20. A graminaceous plantlet regenerated by the method of claim 10.

21. A wheat plantlet regenerated by the method of claim 14.

22. A corn plantlet regenerated by the method of claim 16.
